(19) United States
(12) Patent Application Publication
(10) Pub. No.: US 2008/0255080 A1
(43) Pub. Date: Oct. 16, 2008

(54) Hydroquinone Ansamycin Formulations

(76) Inventors: James L. Wright, Lexington, MA (US); James R. Porter, Rowley, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/101,473

(22) Filed: Apr. 11, 2008

Related U.S. Application Data
(60) Provisional application No. 60/911,330, filed on Apr. 12, 2007.

(51) Int. Cl. A61K 31/395; A61P 35/00; A61P 35/02

(52) U.S. Cl. 514/183

(57) ABSTRACT

Pharmaceutical compositions of hydroquinone geldanamycin analogs, and uses of such compositions, are provided.

### RELATED APPLICATIONS

This application claims benefit under 35 U.S.C. 119(e) of the filing date of U.S. Ser. No. 60/911,330, filed on Apr. 12, 2007, the entire disclosure of which is incorporated herein by reference.

### BACKGROUND

Heat shock protein 90 (Hsp90) is a highly abundant mammalian protein, which is essential for cell viability and which exhibits dual chaperone functions. It plays a key role in the cellular stress-response by interacting with proteins after their native conformations have been altered by various environmental stresses, such as heat shock, thereby ensuring adequate protein-folding and preventing non-specific aggregation. Hsp90 may also play a role in buffering proteins against the effects of mutation, presumably by correcting the inappropriate folding of mutant proteins. Hsp90 also has an important regulatory role under normal physiological conditions and it is responsible for the conformational stability and maturation of a number of specific client proteins.

Hsp90 antagonists are currently being explored in a large number of biological contexts where a therapeutic effect may be obtained for a condition or disorder by inhibiting one or more aspects of Hsp90 activity. Although the primary focus of the research has been on proliferative disorders, such as cancers, other conditions have also been shown to be amenable to treatment using Hsp90 antagonists.

Geldanamycin's nanomolar potency and apparent selectivity for killing tumor cells, as well as the discovery that its primary target in mammalian cells is Hsp90, has stimulated interest in its development as an anti-cancer drug. However, the extremely low water solubility of geldanamycin and the association of hepatotoxicity with its administration have led to difficulties in developing an approvable agent for therapeutic applications. Thus, a need exists for analogs of geldanamycin that may be developed as potential therapeutics, and for formulations of these compounds that may be delivered to patients.

### DETAILED DESCRIPTION

### Definitions

The definitions of terms used herein are meant to incorporate the present state-of-the-art definitions recognized for each term in the chemical and pharmaceutical fields. Where appropriate, exemplification is provided. The definitions apply to the terms as they are used throughout this specification, unless otherwise limited in specific instances, either individually or as part of a larger group.

Where stereochemistry is not specifically indicated, all stereoisomers of the inventive compounds are included within the scope of this disclosure, as pure compounds (i.e., stereoisomers) as well as mixtures thereof. Unless otherwise indicated, individual enantiomers, diastereomers, geometrical isomers, and combinations and mixtures thereof are all encompassed by the present disclsosure. Polymorphic crystalline forms and solvates are also encompassed within the scope of this disclosure.

The term “acylamino” is art-recognized and refers to a moiety that may be represented by the general formula:

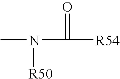

wherein R50 is as defined above, and R54 represents a hydrogen, an alkyl, an alkenyl or —(CH₂)ₘ—R61, where m and R61 are as defined above.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has 30 or fewer carbon atoms in its backbone (e.g., C₁-C₃₀ for straight chain, C₃-C₃₀ for branched chain), 20 or fewer. Likewise, certain cycloalkyls have from 3-10 carbon atoms. In some embodiments, an alkyl group contains 1-10 carbon atoms as its backbone, and may be substituted. Likewise, certain cycloalkyls have from 3-10 carbon atoms in their ring structure, and others have 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “aralkyl” is art-recognized and refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively. Alkenyl and alkynyl groups may be substituted with the same groups that are suitable as substituents on alkyl groups, to the extent permitted by the available valences. Typical alkenyl and alkynyl groups contain 2-10 carbons in the backbone structure.

The terms ““alkoxyl”” or ““alkoxy”” refers to an alkyl group, as defined above, having an oxygen radical attached thereto. Representative alkoxyl groups include methoxy, ethoxy, propyloxy, tert-butoxy and the like. The alkyl portion of an alkoxy group is sized like the alkyl groups, and may be substituted by the same groups that are suitable as substituents on alkyl groups, to the extent permitted by the available valences.

The term “acyl” as used herein refers to a group of the general formula R—C(═O)—, where R may be H, alkyl, aryl, or aralkyl. In typical acyl groups, R is H or C1-C6 alkyl, which is optionally substituted, or R may be aralkyl, wherein the aryl portion of the aralkyl is a 5-7 membered aromatic or heteroaromatic ring, and the alkyl portion is a C1-C4 alkylene group; and both the alkyl and aryl portions are optionally substituted as described herein for such groups. Benzyl, p-methoxybenzyl, and phenylethyl are examples of a typical aralkyl.

The term “amido” and “amide” are art recognized as an amino-substituted carbonyl and includes a moiety that may be represented by the general formula:

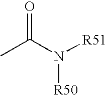

wherein R50 and R51 are as defined above.

The terms “amine” and “amino” are art-recognized and refer to both unsubstituted and substituted amines, e.g., a moiety that may be represented by the general formulas:

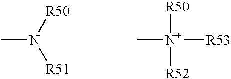

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “aralkyl” as used herein, whether alone or as part of a group name such as, for example, aralkyloxy, refers to an alkyl group as described herein substituted with an aryl group as described herein (e.g., an aromatic or heteroaromatic group). Both the alkyl and the aryl portion of each aralkyl group are typically optionally substituted. Typical aralkyl groups include, for example, groups of general formula Ar—(CH₂)ₜ—, where Ar represents an aromatic or heteroaromatic ring and t is an integer from 1-6.

The term “aryl” as used herein, whether alone or as part of another name, such as ‘aryloxy’, refers to 5-, 6- and 7-membered single-ring aromatic groups that may include from zero to four heteroatoms selected from N, O and S, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

As used herein, the term “benzoquinone ansamycin” refers to a compound comprising a macrocyclic lactam, further comprising only one amide in the lactam ring and a benzoquinone moiety in the lactam ring, wherein said benzoquinone moiety has at least one nitrogen substituent, wherein one of said at least one nitrogen substitutents is part of said only one amide moiety in the lactam ring. Specific examples of naturally-occurring benzoquinone ansamycins include, but are not limited to, geldanamycin and herbimycin. In the corresponding “hydroquinone ansamycin,” the benzoquinone moiety is reduced to a hydroquinone.

The term ‘heterocycloalkyl’ refers to cycloalkyl groups as described herein, wherein at least one carbon atom of the alkyl or cycloalkyl portion is replaced by a heteroatom selected from N, O and S.

The terms “heterocyclyl”, “heteroaryl”, “heterocyclic ring” or “heterocyclic group” are art-recognized and refer to 3-membered to about 10-membered ring structures, alternatively 3-membered to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “Hsp90 mediated disorder” or “disorder mediated by cells expressing Hsp90” refers to pathological and disease conditions in which Hsp90 plays a role. Such roles may be directly related to the pathological condition or may be indirectly related to the condition. The common feature to this class of conditions is that they may be ameliorated by inhibiting the activity, function, or association with other proteins of Hsp90.

The term “hydrogen bond donor” refers to an excipient, containing at least one —OH moiety, that is capable of forming at least one hydrogen bond with the hydroquinone ansamycin, thereby stabilizing the hydroquinone ansamycin in the solid state. In some embodiments, the hydrogen bond donor contains more than one —OH moiety. The compounds ascorbic acid and citric acid are specifically excluded from the group of excipients that are considered “hydrogen bond donors.”

As used herein, the term “isolated” in connection with a compound provided herein means the compound is not in a cell or organism and the compound is separated from some or all of the components that typically accompany it in nature.

The term “nitro” is art-recognized and refers to —NO₂; the terms “halogen” and “halide” are art-recognized and refers to —F, —Cl, —Br or —I; the term “sulfhydryl” means —SH; and the term “hydroxyl” means —OH. “Halide” designates the corresponding anion of the halogens, and “pseudohalide” has the definition set forth in “Advanced Inorganic Chemistry” by Cotton and Wilkinson.

The term “pharmaceutically acceptable salt” or “salt” refers to a salt of one or more compounds. Suitable pharmaceutically acceptable salts of compounds include acid addition salts which may, for example, be formed by mixing a solution of the compound with a solution of a pharmaceutically acceptable acid, such as hydrochloric acid, hydrobromic acid, sulfuric acid, fumaric acid, maleic acid, succinic acid, benzoic acid, acetic acid, citric acid, tartaric acid, phosphoric acid, carbonic acid, or the like. Where the compounds carry one or more acidic moieties, pharmaceutically acceptable salts may be formed by treatment of a solution of the compound with a solution of a pharmaceutically acceptable base, such as lithium hydroxide, sodium hydroxide, potassium hydroxide, tetraalkylammonium hydroxide, lithium carbonate, sodium carbonate, potassium carbonate, ammonia, alkylamines, or the like.

The term “pharmaceutically acceptable carrier” refers to a medium that is used to prepare a desired dosage form of a compound. A pharmaceutically acceptable carrier can include one or more solvents, diluents, or other liquid vehicles; dispersion or suspension aids; surface active agents; isotonic agents; thickening or emulsifying agents; preservatives; solid binders; lubricants; and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe ed. (American Pharmaceutical Assoc. 2000), disclose various carriers used in formulating pharmaceutical compositions and known techniques for the preparation thereof.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this disclosure.

As used herein, the term “pure” in connection with an isolated sample of a compound provided herein means the isolated sample contains at least about 60% by weight of the compound, at least about 70% by weight of the compound, at least about 80% by weight of the compound, at least about 90% by weight of the compound, or at least about 95% by weight of the compound. The purity of an isolated sample of a compound provided herein may be assessed by any of a number of methods or a combination of them; e.g., thin-layer, preparative or flash chromatography, mass spectrometry, HPLC, NMR analysis, and the like.

The term “subject” as used herein, refers to an animal, typically a mammal or a human, that will be or has been the object of treatment, observation, and/or experiment. When the term is used in conjunction with administration of a compound or drug, then the subject has been the object of treatment, observation, and/or administration of the compound or drug.

The term “substituted” refers to a chemical group, such as alkyl, cycloalkyl aryl, and the like, wherein at least one hydrogen is replaced with a with a substituent as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this disclosure, the heteroatoms, such as nitrogen, may have hydrogen substituents and/or any permissible substituents of organic compounds described herein which satisfy the valences of the heteroatoms. This disclosure is not intended to be limited in any manner by the permissible substituents of organic compounds.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation, such as by rearrangement, cyclization, elimination, or other reaction.

The definition of each expression, e.g., alkyl, m, n, and the like, when it occurs more than once in any structure, is intended to be independent of its definition elsewhere in the same structure.

The term “sugar” as used herein refers to a natural or an unnatural monosaccharide, disaccharide, oligosaccharide, or polysaccharide, comprising one or more triose, tetrose, pentose, hexose, heptose, octose, or nonose saccharides. Sugars may include substances derived from saccharides by reduction of the carbonyl group (alditols), by oxidation of one or more terminal groups to carboxylic acids (aldonic acids), or by replacement of one or more hydroxyl group(s) by a hydrogen (deoxy sugars), an amino group (amino sugars), a thiol group (thio sugars), an acylamino group, a sulfate group, a phosphate group, or similar heteroatomic group; or any combination of the foregoing modifications. The term sugar also includes derivatives of these compounds (i.e., sugars that have been chemically modified by acylation, alkylation, and formation of glycosidic bonds by reaction of sugar alcohols with aldehydes or ketones, etc). Sugars may be present in cyclic (oxiroses, oxetosesm furanoses, pyranoses, septanoses, octanoses, etc) form as hemiacetals, hemiketals, or lactones; or in acyclic form. The saccharides may be ketoses, aldoses, polyols and/or a mixture of ketoses, aldoses and polyols. Sugars include, but are not limited to glycerol, polyvinylalcohol, propylene glycol, sorbitol, ribose, arabinose, xylose, lyxose, allose, altrose, mannose, mannitol, gulose, dextrose, idose, galactose, talose, glucose, fructose, dextrates, lactose, sucrose, starches (i.e., amylase and amylopectin), sodium starch glycolate, cellulose and cellulose derivatives (i.e., methylcellulose, hydroxypropyl celluloe, hydroxyethyl cellulose, hydroxyethylmethyl cellulose, carboxymethyl cellulose, cellulose acetate, cellulose acetate phthalate, croscarmellose, hypomellose, and hydroxypropyl methyl cellulose), carrageenan, cyclodextrins, dextrin, polydextrose, and trehalose.

The term “therapeutically effective amount” as used herein, means that amount of active compound or pharmaceutical agent that elicits a biological or medicinal response in a cell culture, tissue system, animal, or human that is being sought by a researcher, veterinarian, clinician, or physician, which includes alleviation of the symptoms of the disease, condition, or disorder being treated.

Where two groups “taken together form a bond,” if the groups are attached to atoms that are not otherwise directly bonded to each other, they represent a bond between the atoms to which they are attached. If the groups are on atoms that are directly bonded to each other, they represent an additional bond between those two atoms. Thus, for example, when R² and R³ taken together form a bond, the structure —C(A)R²—C(B)R³— represents —C(A)═C(B)—.

Certain compounds contained in compositions disclosed herein may exist in particular geometric or stereoisomeric forms. The present disclosure contemplates all such compounds, including cis- and trans-isomers, R- and S-enantiomers, diastereomers, (D)-isomers, (L)-isomers, the racemic mixtures thereof, and other mixtures thereof, as falling within the scope of this disclosure. Additional asymmetric carbon atoms may be present in a substituent such as an alkyl group. All such isomers, as well as mixtures thereof, are intended to be included in this disclosure.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

### Compositions

The compositions disclosed herein comprise a hydrogen bond donor and a hydroquinone ansamycin. In certain embodiments of the compositions, the hydroquinone ansamycin is a compound of formula 1:

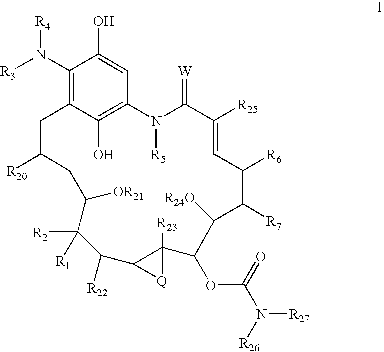

or a pharmaceutically acceptable salt thereof,

wherein independently for each occurrence:

W is oxygen or sulfur;

Q is oxygen, NR, N(acyl) or a bond;

R for each occurrence is independently selected from the group consisting of hydrogen, alkyl, cycloalkyl, heterocycloalkyl, aralkyl, heteroaryl, and heteroaralkyl;

R₁ is hydroxyl, alkoxyl, —OC(O)R₈, —OC(O)OR₉, —OC(O)NR₁₀R₁₁, —OSO₂R₁₂, —OC(O)NHSO₂NR₁₃R₁₄, —NR₁₃R₁₄, or halide; and R₂ is hydrogen, alkyl, or aralkyl; or R₁ and R₂ taken together, along with the carbon to which they are bonded, represent —(C═O)—, —(C═N—OR)—, —(C═N—NHR)—, or —(C═N—R)—;

R₃ and R₄ are each independently selected from the group consisting of hydrogen, alkyl, alkenyl, alkynyl, aryl, cycloalkyl, heterocycloalkyl, aralkyl, heteroaryl, heteroaralkyl, and —[(C(R)₂)ₚ]—R₁₆; or R₃ taken together with R₄ represent a 4-8 membered optionally substituted heterocyclic ring;

R₅ is selected from the group consisting of H, alkyl, aralkyl, and a group having the formula 1a:

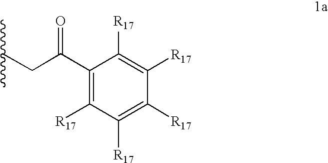

wherein each occurrence of R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₈), and —CH₂O-heterocyclyl;

R₆ and R₇ are both hydrogen; or R₆ and R₇ taken together form a bond;

R₈ is hydrogen, alkyl, alkenyl, alkynyl, aryl, cycloalkyl, heterocycloalkyl, aralkyl, heteroaryl, heteroaralkyl, or —[(C(R)₂)ₚ]—R₁₆;

R₉ is alkyl, alkenyl, alkynyl, aryl, cycloalkyl, heterocycloalkyl, aralkyl, heteroaryl, heteroaralkyl, or —[(C(R)₂)ₚ]—R₁₆;

R₁₀ and R₁₁ are each independently selected from the group consisting of hydrogen, alkyl, alkenyl, alkynyl, aryl, cycloalkyl, heterocycloalkyl, aralkyl, heteroaryl, heteroaralkyl, and —[(C(R)₂)ₚ]—R₁₆; or R₁₀ and R₁₁ taken together with the nitrogen to which they are bonded represent a 4-8 membered optionally substituted heterocyclic ring;

R₁₂ is alkyl, alkenyl, alkynyl, aryl, cycloalkyl, heterocycloalkyl, aralkyl, heteroaryl, heteroaralkyl, or —[(C(R)₂)ₚ]—R₁₆;

R₁₃ and R₁₄ are each independently selected from the group consisting of hydrogen, alkyl, alkenyl, alkynyl, aryl, cycloalkyl, heterocycloalkyl, aralkyl, heteroaryl, heteroaralkyl, and —[(C(R)₂)ₚ]—R₁₆; or R₁₃ and R₁₄ taken together with the nitrogen to which they are bonded represent a 4-8 membered optionally substituted heterocyclic ring;

R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), OS(O)₂OR₁₈, —S(O)₂OR₁₈, —OP(O)(OR₁₈)(OR₁₉), —N(R₁₈)P(O)(OR₁₈)(OR₁₉), and —P(O)(OR₁₈)(OR₁₉);

p is 1, 2, 3, 4, 5, or 6;

R₁₈ for each occurrence is independently selected from the group consisting of hydrogen, alkyl, aryl, cycloalkyl, heterocycloalkyl, aralkyl, heteroaryl, and heteroaralkyl;

R₁₉ for each occurrence is independently selected from the group consisting of hydrogen, alkyl, aryl, cycloalkyl, heterocycloalkyl, aralkyl, heteroaryl, and heteroaralkyl; or R₁₈ taken together with R₁₉ represent a 4-8 membered optionally substituted ring;

R₂₀, R₂₁, R₂₂, R₂₄, and R₂₅, for each occurrence are independently alkyl;

R₂₃ is alkyl, —CH₂OH, —CHO, —COOR₁₈, or —CH(OR₁₈)₂;

R₂₆ and R₂₇ for each occurrence are independently selected from the group consisting of hydrogen, alkyl, aryl, cycloalkyl, heterocycloalkyl, aralkyl, heteroaryl, and heteroaralkyl;

the absolute stereochemistry at a stereogenic center of formula 6 is R or S or a mixture thereof and the stereochemistry of a double bond is E or Z or a mixture thereof.

In certain embodiments, the compositions contain pure, isolated and/or pure and isolated compound 1.

The compositions have a hydrogen bond donor component. The hydrogen bond donor component can include any pharmaceutically acceptable excipient that is capable of forming at least one hydrogen bond with the hydroquinone ansamycin, thereby stabilizing the hydroquinone ansamycin in the solid state and minimizing oxidation to the corresponding benzoquinones. Sugars contain multiple —OH groups and are therefore exemplary hydrogen bond donors. Specific examples of sugars include glycerol, glycerol monostearate, polyvinylalcohol, propylene glycol, sorbitol, ribose, arabinose, xylose, lyxose, allose, altrose, mannose, mannitol, gulose, dextrose, idose, galactose, talose, glucose, fructose, dextrose, dextrates, lactose, sucrose, maltose, starches (e.g., corn starch, amylase, amylopectin), sodium starch glycolate, cellulose and cellulose derivativees (i.e., methylcellulose, hydroxypropyl cellulose, hydroxyethyl cellulose, hydroxyethylmethyl cellulose, carboxymethyl cellulose, cellulose acetate, cellulose acetate phthalate, croscarmellose, hypromellose, and hydroxypropyl methyl cellulose), carrageenan, cyclodextrins, dextrin, polydextrose, malic acid, trehalose, and derivatives of any of the above. Non-sugar examples include stearic acid and Vitamin E. The presence of the hydrogen bond donor stabilizes the hydroquinone ansamycins for extended periods of time. The ratio of the hydrogen bond donor to the hydroquinone ansamycin may be about 1:99 to about 99:1, about 1:19 to about 19:1, about 1:9 to about 7:3, about 1:4 to about 1:1, or about 3:7 to about 1:1.2 (weight/weight).

The compounds described above may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts may be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. See, for example, Berge et al. (1977) “Pharmaceutical Salts”, J. Pharm. Sci. 66:1-19.

Pharmaceutically acceptable salts include the conventional non-toxic salts or quaternary ammonium salts of the compounds, e.g., from non-toxic organic or inorganic acids. For example, such conventional non-toxic salts include those derived from inorganic acids, such as hydrochloride, hydrobromic, sulfuric, sulfamic, phosphoric, nitric, and the like; and salts prepared from organic acids, such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

The compositions may also contain an anti-oxidant, such as ascorbate, cysteine hydrochloride, sodium bisulfate, sodium metabisulfite, sodium sulfite, thioglycerol, sodium mercaptoacetate, sodium formaldehyde sulfoxylate, ascorbyl palmitate, butylated hydroxyanisole, butylated hydroxytoluene, lecithin, propyl gallate, or alpha-tocopherol. The molar ratio of the antioxidant to the hydroquinone ansamycin may be between about 0.001:1 to about 4:1, about 0.01:1 to about 3:1, about 0.1:1 to about 2:1, about 1:1 to 2:1, about 1:1 to about 1.5:1, or about 1:1.5 to about 1:1.

In the examples below, a general procedure is described for preparing ansamycin hydroquinone pharmaceutical compositions, comprising contacting an ansamycin hydroquinone with a solution containing ascorbic acid and trehalose; this procedure may be used with any of the hydrogen bond donors described herein or any of the anti-oxidants described herein, or both. The resulting solution is then lyophilized to prepare a lyo-powder.

The compositions may also contain metal chelators, such as citric acid, ethylenediamine tetraacetic acid (EDTA) or a salt thereof, DTPA (diethylene-triamine-penta-acetic acid) or a salt thereof, EGTA or a salt thereof, NTA (nitriloacetic acid) or a salt thereof, sorbitol or a salt thereof, tartaric acid or a salt thereof, N-hydroxy iminodiacetate or a salt thereof, hydroxyethyl-ethylene diamine-tetraacetic acid or a salt thereof, 1- or 3-propanediamine tetra acetic acid or a salt thereof, 1- or 3-diamino-2-hydroxy propane tetra-acetic acid or a salts thereof, sodium gluconate, hydroxy ethane diphosphonic acid or a salt thereof, or phosphoric acid or a salt thereof.

The compositions may also contain one or more wetting agents, emulsifiers and lubricants (e.g., sodium lauryl sulfate or magnesium stearate), coloring agents, release agents, coating agents, sweetening, flavoring and perfuming agents, preservatives, solubilizing agents, and buffering agents (e.g., citrate, ascorbate, phosphate, bicarbonate, carbonate, fumarate, acetate, tartarate or malate), solubilizing agents (e.g., polyoxyethylene sorbitan fatty acid esters, polyoxyethylene stearates, benzyl alcohol, ethyl alcohol, polyethylene glycols, propylene glycol, glycerin, cyclodextrin, or poloxamers), or complexing agents (e.g., cyclodextrins, especially substituted beta cyclodextrins, such as 2-hydroxypropyl-beta, dimethyl beta, 2-hydroxyethyl beta, 3-hydroxypropyl beta, and trimethyl beta).

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

In some embodiments, the composition is an amorphous powder. Excipients that stabilize the amorphous powder, such as polyethylene glycol (PEG), and polyvinylpyrrolidone (PVP), can be added as well.

### Methods for Making Compounds

A variety of methodologies may be adapted for generating the compounds disclosed herein. In general, the steps involve (1) converting an ansamycin to a 17-demethoxy-17-amino analog (e.g., 17-AG or 17-AAG), (2) reducing the benzoquinone in the ansamycin to give a hydroquinone, (3) optionally forming a salt of the hydroquinone, and (4) combining the compound/salt with a hydrogen bond donor and optionally with one or more other components, as outlined above.

A benzoquinone-containing ansamycin may be obtained via fermentation of a strain producing the compound (for example, see WO 03/072794 and U.S. Pat. No. 3,595,955). Alternatively, synthetic or semi-synthetic methodology may be used to produce the ansamycin (see U.S. Pat. No. 5,387,584 and WO 00/03737). Further, there are commercial suppliers of isolated fermentation materials, such as geldanamycin; therefore, such materials are readily available.

For example, geldanamycin may be isolated from a fermentation culture of an appropriate micro-organism and may be derivatized using a variety of functionalization reactions known in the art. Representative examples include metal-catalyzed coupling reactions, oxidations, reductions, reactions with nucleophiles, reactions with electrophiles, pericyclic reactions, installation of protecting groups, removal of protecting groups, and the like. Many methods are known in the art for generating analogs of the various benzoquinone ansamycins (for examples, see U.S. Pat. Nos. 4,261,989; 5,387,584; and 5,932,566 and J. Med. Chem. 1995, 38, 3806-3812, herein incorporated by reference).

A variety of methods and reaction conditions may be used to reduce the benzoquinone portion of the ansamycin. Sodium hydrosulfite may be used as the reducing agent. Other reducing agents that may be used include, but are not limited to, zinc dust with acetic anhydride or acetic acid, ascorbic acid and electrochemical reductions. Typically, the geldanamycin analog is dissolved in an organic solvent, such as EtOAc. Other solvents that may be used include, but are not limited to, dichloromethane, chloroform, dichloroethane, chlorobenzene, THF, MeTHF, diethyl ether, diglyme, 1,2-dimethoxyethane, MTBE, THP, dioxane, 2-ethoxybutane, methyl butyl ether, methyl acetate, 2-butanone, water and mixtures thereof. Two or more equivalents of sodium hydrosulfite are then added as a solution in water (5-30% (m/v), or for example 10% (m/v)), to the reaction vessel at room temperature. Aqueous solutions of sodium hydrosulfite are unstable and therefore need to be freshly prepared prior to use. Vigorous mixing of the biphasic mixture ensures reasonable reaction rates.

Upon completion of the reduction, the crude reaction product may be used directly (i.e., without purification) in the preparation of the pharmaceutical compositions provided herein, thereby minimizing oxidation of the hydroquinone.

The hydroquinones provided herein may be converted into salt form by reaction with an acid, or by reaction with an acid halide of an amino acid. In the examples, the C-17 alkyl amino group is protonated to generate a C-17 ammonium salt hydroquinone geldanamycin analog. In addition, the C-17 ammonium salt hydroquinones formed have the added benefit of being highly soluble in aqueous solutions (solubility >200 mg/mL), unlike 17-AAG (solubility <100 μg/mL).

The ammonium salt of the hydroquinone is formed by the addition of a solution of an acid, such as HCl, in an organic solvent, such as EtOAc, DCM, IPA or dioxane, to the hydroquinone containing ansamycin in an organic solution; the organic solvents may be independently acetone, dichloromethane, chloroform, dichloroethane, chlorobenzene, THF, MeTHF, diethyl ether, diglyme, 1,2-dimethoxyethane, MTBE, THP, dioxane, 2-ethoxybutane, methyl butyl ether, methyl acetate, or 2-butanone, under an atmosphere of nitrogen or other inert gas or a mixture of inert gases.

The ammonium salt of the hydroquinone is collected by filtration in cases where the product precipitates from solution. In cases where the ammonium salt hydroquinone does not precipitate, the reaction solution is concentrated under reduced pressure to yield the product. A variety of ammonium salt hydroquinone ansamycins may be synthesized by using organic or inorganic acids. Some acids that may be used include, but are not limited to HCl, HBr, H₂SO₄, methansulfonic acid, benzenesulfonic acid, p-toluenesulfonic acid, triflic acid, camphorsulfonic acid, naphthalene-1,5-disulfonic acid, ethan-1,2-disulfonic acid, cyclamic acid, thiocyanic acid, naphthalene-2-sulfonic acid, oxalic acid, and the like. See, for example, Berge et al. (1977) “Pharmaceutical Salts”, J. Pharm. Sci. 66:1-19. Any acid with a pKa between about −10 and about 7, about −10 and about 4, between about −10 and about 1, and between about −10 and about −3 may be used to generate the ammonium salt hydroquinone.

### Methods for Making Compositions

Compositions may be made using the following procedure: distilled water is chilled in an ice-water bath; argon may be bubbled through the solution. A hydrogen bond donor can then be added and allowed to dissolve. An anti-oxidant and any other additional components can then be added. Once all of the solids have dissolved, the hydroquinone ansamycin is added and the ice-water bath is removed. When the solids are completely dissolved, the solution is lyophilized or spray dried. The resulting powder is then stored under argon.

The pharmaceutical compositions disclosed herein may be specially formulated for administration in solid or liquid form, for example, tablets, capsules, drenches (aqueous or non-aqueous solutions or suspensions), powders, granules, or pastes.

### Administration of Compositions

When the pharmaceutical compositions disclosed herein are used as antiproliferative agents, such as anticancer agents, they may be administered alone or in combination with an additional pharmaceutically acceptable carrier or diluent in a pharmaceutical composition according to standard pharmaceutical practice. The compositions may be administered orally or parenterally. Parenteral administration includes intravenous, intramuscular, intraperitoneal, subcutaneous and topical administration.

### Pharmaceutical Uses and Methods of Treatment

Also provided herein are methods of treating cancer, inhibiting Hsp90, and/or treating a hyperproliferative disorder comprising orally administering to a patient in need thereof a therapeutically effective amount of any of the aforementioned compounds or pharmaceutical compositions. The hydroquinone-containing compounds disclosed herein rapidly oxidize to 17-amino substituted benzoquinone geldanamycin analogs (e.g., 17-AAG) in vitro and in vivo at physiological pH. As such, the hydroquinone analogs exhibit similar biological activities and therapeutic profiles as do 17-amino substituted geldanamycin analogs and may be used for all known therapeutic indications against which 17-amino substituted geldanamycin analogs are useful. 17-Amino-substituted geldanamycin analogs, in particular 17-AG and 17-AAG, are highly potent and selective inhibitors of Hsp90. The cancer, neoplastic disease state or hyperproliferative disorder is selected from the group consisting of gastrointestinal stromal tumor (GIST), colon cancer, colorectal cancer, pancreatic cancer, breast cancer, ovarian cancer, prostate cancer, small cell lung cancer, non-small cell lung cancer, melanoma, multiple myeloma, myelodysplastic syndrome, acute lymphocytic leukemia, acute myelocytic leukemia, chronic myelocytic leukemia, chronic lymphocytic leukemia, polycythemia Vera, Hodgkin lymphoma, non-Hodgkin lymphoma, Waldenstrom's macroglobulinemia, heavy chain disease, soft-tissue sarcomas, such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, stadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, endometrial cancer, follicular lymphoma, diffuse large B-cell lymphoma, mantle cell lymphoma, hepatocellular carcinoma, thyroid cancer, gastric cancer, esophageal cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, thyroid cancer, neuroendocrine cancers, and carcinoid tumors.

In certain embodiments, the cancer is selected from the group consisting of gastrointestinal stromal tumor, multiple myeloma, prostate cancer, breast cancer, melanoma, chronic myelocytic leukemia, and non-small cell lung cancer.

Actual dosage levels of the hydroquinone ansamycins in the pharmaceutical compositions may be varied so as to obtain an amount of the compound which is effective to achieve the desired therapeutic response for a particular patient, composition, and mode of administration, without being toxic to the patient.

The selected dosage level will depend upon a variety of factors including the activity of the particular geldanamycin analog employed, or salt thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts. The administered dose can be between 10 mg and 2000 mg, or between 50 mg and 1500 mg, or between 100 mg and 800 mg. For example, a dose can be 700 mg. The dose can be administered, e.g., in 100 and 200 mg tablets or capsules.

The composition can be administered daily, every other day, three times a week, twice a week, weekly, or bi-weekly. The dosing schedule can include a “drug holiday,” i.e., the drug can be administered for two weeks on, one week off, or continuously, without a drug holiday.

### Combination Therapy

In some embodiments, the pharmaceutical compositions described herein can be used in combination with other therapeutic agents in order to achieve selective activity in the treatment of cancer. In certain embodiments, the geldanamycin analogs described herein are used to reduce the cellular levels of properly folded Hsp90 client proteins, which are then effectively inhibited by the second agent. For example, binding of a benzoquinone ansamycin analog to Hsp90 results in targeting of the client protein to the proteasome, and subsequent degradation. Using an agent that targets and inhibits the proteasome, e.g., Velcade®, then leads to increased cellular apoptosis and cell death.

Some examples of therapeutic agents which can be used in combination with the formulations described herein include alkylating agents; anti-angiogenic agents; anti-metabolites; epidophyllotoxin; procarbazine; mitoxantrone; platinum coordination complexes; anti-mitotics; biological response modifiers and growth inhibitors; hormonal/anti-hormonal therapeutic agents; haematopoietic growth factors; the anthracycline family of drugs; the vinca drugs; the mitomycins; the bleomycins; the cytotoxic nucleosides; the epothilones; discodermolide; the pteridine family of drugs; diynenes; and the podophyllotoxins. Particularly useful members of those classes include, for example, caminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, doxorubicin, vindesine, leurosine, paclitaxel, taxol, taxotere, docetaxel, cis-platin, imatinib mesylate, or gemcitebine.

Other useful agents include estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins. Particularly useful agents include taxotere, Gleevec (imatinib), Tarceva (erlotinib), Sutent (sunitinib), Tykerb (lapatinib), and Xeloda (capecitabine).

The formulations described herein can also be used in conjunction with radiation therapy. The chemotherapeutic agent/radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. The therapeutic protocols (e.g., dosage amounts and times of administration) can be varied in view of the observed effects of the administered therapeutic agents (i.e., antineoplastic agent or radiation) on the patient, and in view of the observed responses of the disease to the administered therapeutic agents.

Also, in general, the geldanamycin analogs described herein and the second chemotherapeutic agent do not have to be administered in the same pharmaceutical composition, and may, because of different physical and chemical characteristics, have to be administered by different routes. For example, the geldanamycin compound can be administered orally, while the second chemotherapeutic is administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of chemotherapeutic agent or radiation will depend upon the diagnosis of the attending physicians and their judgment of the condition of the patient and the appropriate treatment protocol.

The geldanamycin analog and the second chemotherapeutic agent and/or radiation may be administered concurrently (e.g., simultaneously, essentially simultaneously or within the same treatment protocol) or sequentially, depending upon the nature of the proliferative disease, the condition of the patient, and the actual choice of chemotherapeutic agent and/or radiation to be administered in conjunction (i.e., within a single treatment protocol) with the geldanamycin analog.

If the geldanamycin analog, and the chemotherapeutic agent and/or radiation are not administered simultaneously or essentially simultaneously, then the optimum order of administration may be different for different tumors. Thus, in certain situations the geldanamycin analog may be administered first followed by the administration of the chemotherapeutic agent and/or radiation; and in other situations the chemotherapeutic agent and/or radiation may be administered first followed by the administration of a geldanamycin analog. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the chemotherapeutic agent and/or radiation may be administered first, especially if it is a cytotoxic agent, and then the treatment continued with the administration of a geldanamycin analog followed, where determined advantageous, by the administration of the chemotherapeutic agent and/or radiation, and so on until the treatment protocol is complete.

Thus, in accordance with experience and knowledge, the practicing physician can modify each protocol for the administration of a component (therapeutic agent, i.e., geldanamycin analog, chemotherapeutic agent or radiation) of the treatment according to the individual patient's needs, as the treatment proceeds.

When the geldanamycin analogs are administered in combination with another chemotherapeutic or with radiation, the doses of each agent will in most instances be lower than the corresponding dose for single-agent therapy.

### EXAMPLES

The invention now being generally described, it will be more readily understood by reference to the following examples, which are included merely for purposes of illustration of certain aspects and embodiments of the present invention, and are not intended to limit the invention.

### Example 1

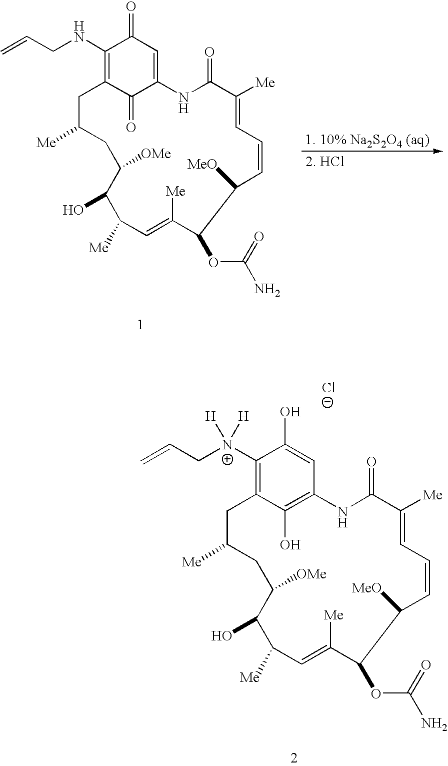

Compound 1 (0.450 g, 0.768 mmol, 1.0 eq) was dissolved in DCM (50 mL) and stirred with a 10% aqueous solution of sodium hydrosulfite (50 mL). The solution was stirred for 30 min. The organic layer was collected, dried over Na₂SO₄, filtered and transferred to a round bottom flask. To this solution was added a solution of HCl in dioxane (4 N, 0.211 mL, 1.1 eq). The resulting mixture was allowed to stir under nitrogen for 30 min. A yellow solid slowly crashed out of solution. The yellow solid was purified by recrystallization form MeOH/EtOAc to yield 0.386 g of compound 2.

### Example 2

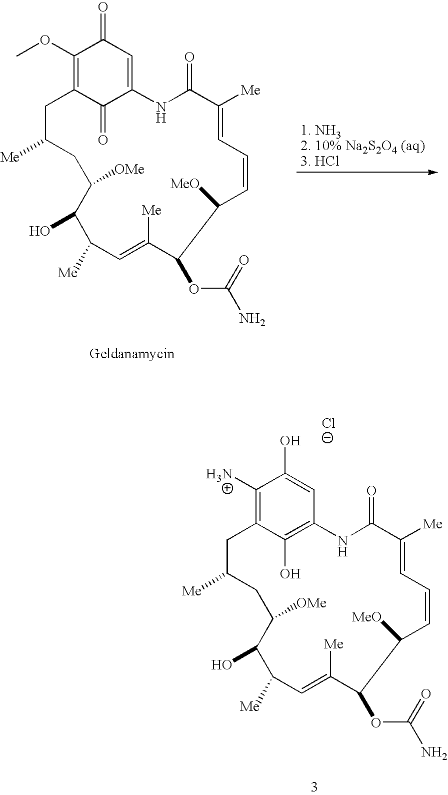

Geldanamycin (1.12 g, 2 mmol, 1 eq) was added to anhydrous DCM (5 mL). NH₃ in MeOH was added to this solution (9 mL, 100 mmol, 50 eq) and was allowed to stir for 24 h. At which point the reaction solution was diluted with DCM and extracted with water, followed by dilute HCl. The organic layer was collected washed with brine, dried over Na₂SO₄ and concentrated to yield a purple solid. This solid was recrystallized twice from acetone/heptanes to yield 0.239 of 17-amino-17-demethoxygeldanamycin.

17-amino-17-demethoxygeldanamycin (0.55 g, 1 mmol, 1 eq) was dissolved in EtOAc (100 mL). A freshly prepared solution of 10% aqueous sodium hydrosulfite (10 mL) was added and stirred for 1 h at rt. The color changed from dark purple to bright yellow, indicating a complete reaction. The layers were separated and the organic phase was dried with magnesium sulfate. The drying agent was rinsed with EtOAc (2×10 mL). The combined filtrate was acidified with 1.5 M HCl in EtOAc (1 mL) to pH 2 over 20 min. The resulting slurry was stirred for 1.5 h at rt. The solids were isolated by filtration, rinsed with ethyl acetate (10 mL) and dried under vacuum to yield the product (0.524 g, 87% yield).

### Example 3

A 2 L round-bottomed flask was charged with a magnetic stir bar, distilled water (426 mL) and cap with a rubber septum. The solution was chilled in an ice-water bath and argon was bubbled through the solution for 20 minutes. The solution was then stirred at 0° C. under an argon atmosphere.

To the solution was added D (+) trehalose dihydrate (25.6 g, 67.7 mmol, 40% wt/wt, calculated by weight of the entire solids). The trehalose powder took about 30 seconds to completely dissolve. To the solution was added L-ascorbic acid (8.4 g, 47.9 mmol, 1.0 eq, based on the amount of hydroquinone ansamycin). The ascorbic acid solid took about 2.5 minutes to dissolve completely.

To the clear solution was added the hydroquinone ansamycin 2 as a solid (29.9 g, 47.9 mmol, 1.0 eq) and the ice-water bath was removed. The solids took approximately 5-10 minutes to dissolve completely.

The solution became more viscous and bubbly and turned a light pink color. When the solids were completely dissolved, the pink solution was transferred to a 1.8 L lyophilizer tray.

An additional 50 mL distilled water was used to rinse the flask and its contents into the tray. The lyophilizer was run according to the following cycle: Segment 1—Prefreeze: −34° C. for 0.5 h; Segment 2—Primary Dry: −20° C. for 15 h (under vacuum); Segment 3—Secondary Dry: 0° C. for 30 h (under vacuum); Segment 4—Hold: 20° C. for 24 h (under vacuum).

The lyophilizer tray was removed from the lyophilizer and 58.0 g of a yellow powder was isolated.

Compositions containing Compound 2 and the excipients, respectively, corn starch, glyceryl monostearate, dextrose, fructose, cellulose, maltose, mannitol, Vitamin E succinate, stearic acid, and lactose monohydrate were made using the procedure described above. All of the compositions, as well as the composition containing trehalose, were stored at room temperature for 4 weeks. At the end of this period, none of the compositions contained more than 5% by weight 17-AAG, thus demonstrating the ability of all of the listed excipients to stabilize the hydroquinone ansamycin.

Similar procedures can be used to make compositions using any of the other hydrogen bond donating excipients disclosed herein, or their equivalents.

### EQUIVALENTS & INCORPORATION BY REFERENCE

It is understood that the examples and embodiments described herein are for illustrative purposes only and that various modifications or changes in light thereof will be suggested to persons skilled in the art and are included within the spirit and purview of this application and scope of the appended claims. All of the U.S. patents and U.S. patent application publications cited herein are hereby incorporated by reference in their entirety for all purposes.

What is claimed is:

1. A pharmaceutical composition, comprising (1) a hydrogen bond donor; and (2) a compound of formula 1: 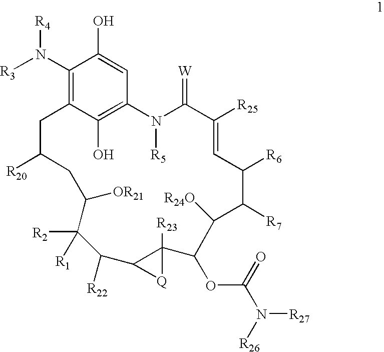
or a pharmaceutically acceptable salt thereof,
wherein independently for each occurrence:
W is oxygen or sulfur;
Q is oxygen, NR, N(acyl) or a bond;
R for each occurrence is independently selected from the group consisting of hydrogen, alkyl, cycloalkyl, heterocycloalkyl, aralkyl, heteroaryl, and heteroaralkyl;
R₁ is hydroxyl, alkoxyl, —OC(O)R₈, —OC(O)OR₉, —OC(O)NR₁₀R₁₁, —OSO₂R₁₂, —OC(O)NHSO₂NR₁₃R₁₄, —NR₁₃R₁₄, or halide; and R₂ is hydrogen, alkyl, or aralkyl; or R₁ and R₂ taken together, along with the carbon to which they are bonded, represent —(C═O)—, —(C═N—OR)—, —(C═N—NHR)—, or —(C═N—R)—;
R₃ and R₄ are each independently selected from the group consisting of hydrogen, alkyl, alkenyl, alkynyl, aryl, cycloalkyl, heterocycloalkyl, aralkyl, heteroaryl, heteroaralkyl, and —[(C(R)₂)ₚ]—R₁₆; or R₃ taken together with R₄ represent a 4-8 membered optionally substituted heterocyclic ring;
R₅ is selected from the group consisting of H, alkyl, aralkyl, and a group having the formula 1a: 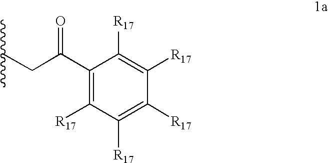
wherein R₁₇ is selected independently from the group consisting of hydrogen, halide, hydroxyl, alkoxyl, aryloxy, acyloxy, amino, alkylamino, arylamino, acylamino, aralkylamino, nitro, acylthio, carboxamide, carboxyl, nitrile, —COR₁₈, —CO₂R₁₈, —N(R₁₈)CO₂R₁₉, —OC(O)N(R₁₈)(R₁₉), —N(R₁₈)SO₂R₁₉, —N(R₁₈)C(O)N(R₁₈)(R₁₉), and —CH₂O-heterocyclyl;
R₆ and R₇ are both hydrogen; or R₆ and R₇ taken together form a bond;
R₈ is hydrogen, alkyl, alkenyl, alkynyl, aryl, cycloalkyl, heterocycloalkyl, aralkyl, heteroaryl, heteroaralkyl, or —[(C(R)₂)ₚ]—R₁₆;
R₉ is alkyl, alkenyl, alkynyl, aryl, cycloalkyl, heterocycloalkyl, aralkyl, heteroaryl, heteroaralkyl, or —[(C(R)₂)ₚ]—R₁₆;
R₁₀ and R₁₁ are each independently selected from the group consisting of hydrogen, alkyl, alkenyl, alkynyl, aryl, cycloalkyl, heterocycloalkyl, aralkyl, heteroaryl, heteroaralkyl, and —[(C(R)₂)ₚ]—R₁₆; or R₁₀ and R₁₁ taken together with the nitrogen to which they are bonded represent a 4-8 membered optionally substituted heterocyclic ring;
R₁₂ is alkyl, alkenyl, alkynyl, aryl, cycloalkyl, heterocycloalkyl, aralkyl, heteroaryl, heteroaralkyl, or —[(C(R)₂)ₚ]—R₁₆;
R₁₃ and R₁₄ are each independently selected from the group consisting of hydrogen, alkyl, alkenyl, alkynyl, aryl, cycloalkyl, heterocycloalkyl, aralkyl, heteroaryl, heteroaralkyl, and —[(C(R)₂)ₚ]—R₁₆; or R₁₃ and R₁₄ taken together with the nitrogen to which they are bonded represent a 4-8 membered optionally substituted heterocyclic ring;
R₁₆ for each occurrence is independently selected from the group consisting of hydrogen, hydroxyl, acylamino, —N(R₁₈)COR₁₉, —N(R₁₈)C(O)OR₁₉, —N(R₁₈)SO₂(R₁₉), —CON(R₁₈)(R₁₉), —OC(O)N(R₁₈)(R₁₉), —SO₂N(R₁₈)(R₁₉), —N(R₁₈)(R₁₉), —OC(O)OR₁₈, —COOR₁₈, —C(O)N(OH)(R₁₈), —OS(O)₂OR₁₈, —S(O)₂OR₈, —OP(O)(OR₁₈)(OR₁₉), —N(R₁₈)P(O)(OR₁₈)(OR₁₉), and —P(O)(OR₁₈)(OR₁₉);
p is 1, 2, 3, 4, 5, or 6;
R₁₈ for each occurrence is independently selected from the group consisting of hydrogen, alkyl, aryl, cycloalkyl, heterocycloalkyl, aralkyl, heteroaryl, and heteroaralkyl;
R₁₉ for each occurrence is independently selected from the group consisting of hydrogen, alkyl, aryl, cycloalkyl, heterocycloalkyl, aralkyl, heteroaryl, and heteroaralkyl; or R₁₈ taken together with R₁₉ represent a 4-8 membered optionally substituted ring;
R₂₀, R₂₁, R₂₂, R₂₄, and R₂₅, for each occurrence are independently alkyl;
R₂₃ is alkyl, —CH₂OH, —CHO, —COOR₁₈, or —CH(OR₁₈)₂;
R₂₆ and R₂₇ for each occurrence are independently selected from the group consisting of hydrogen, alkyl, aryl, cycloalkyl, heterocycloalkyl, aralkyl, heteroaryl, and heteroaralkyl; the absolute stereochemistry at a stereogenic center of formula 6 may be R or S or a mixture thereof, and the stereochemistry of a double bond may be E or Z or a mixture thereof.

2. The composition of claim 1, wherein the hydrogen bond donor is a sugar.

3. The composition of claim 1, wherein the compound of formula 1 is selected from the group consisting of: 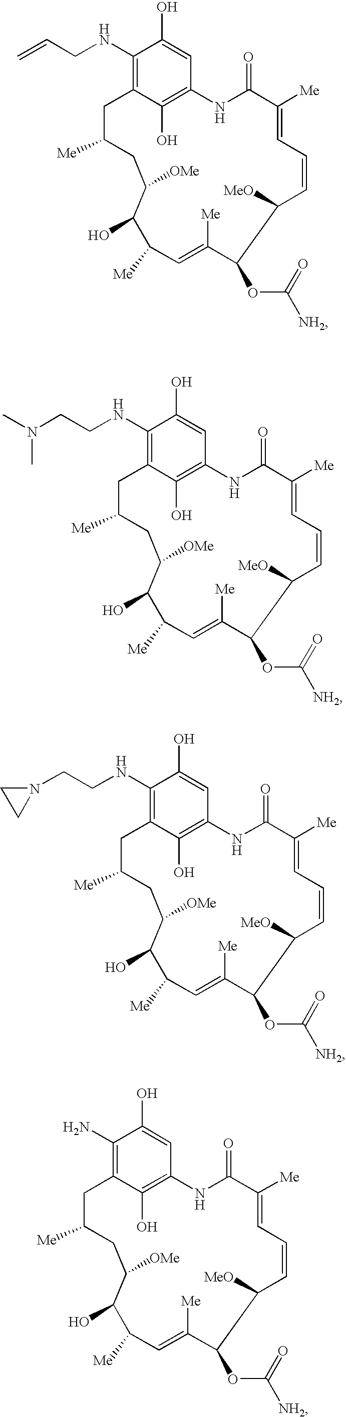 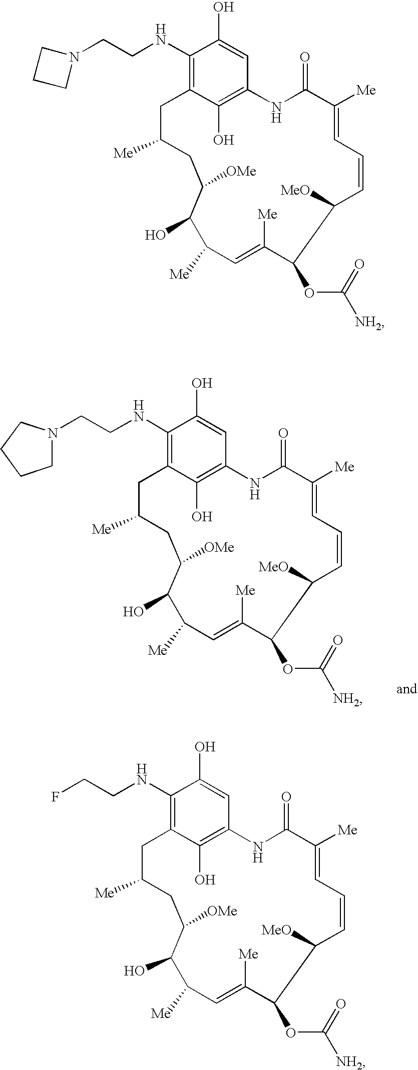
or a pharmaceutically acceptable salt thereof.

4. The composition of claim 1, wherein the hydrogen bond donor is corn starch, glycerol, glyceryl (C1-C20) ester, glucose, fructose, maltose, lactose, trehalose or mannitol.

5. The composition of claim 1, wherein the ratio of the hydrogen bond donor to the hydroquinone ansamycin is about 5:95 to about 99:1 w/w.

6. The composition of any one of claims 1-3, wherein the ratio of the hydrogen bond donor to the hydroquinone ansamycin is about 5:95 to about 80:20 w/w.

7. The composition of any one of claims 1-3, wherein the ratio of the hydrogen bond donor to the hydroquinone ansamycin is about 5:95 to about 40:60 w/w.

8. The composition of claim 1, wherein the hydrogen bond donor is trehalose or mannitol;
and the ratio of trehalose or mannitol to the hydroquinone ansamycin is about 5:95 to about 40:60 w/w.

9. A pharmaceutical composition, comprising (1) trehalose or mannitol; and (2) a hydroquinone ansamycin selected from the group consisting of 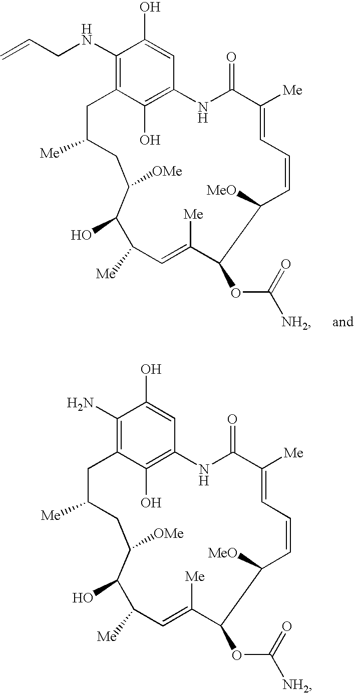
or a pharmaceutically acceptable salt thereof, wherein the ratio of the hydroquinone ansamycin to the trehalose or mannitol is about 5:95 to about 80:20 w/w.

10. The pharmaceutical composition of any one of claims 1-9, further comprising an antioxidant.

11. A method of treating a hyperproliferative disorder, comprising administering to a mammal in need thereof a therapeutically effective amount of a composition of claim 1.

12. The method of claim 11, wherein said mammal is a human.

13. The method of claim 11, wherein said hyperproliferative disorder is breast cancer, multiple myeloma, prostate cancer, Hodgkin lymphoma, non-Hodgkin lymphoma, acute lymphocytic leukemia, chronic lymphocytic leukemia, acute myeloid leukemia, chronic myeloid leukemia, renal cell carcinoma, malignant melanoma, pancreatic cancer, lung cancer, colorectal carcinoma, colon cancer, brain cancer, renal cancer, head and neck cancer, bladder cancer, thyroid cancer, prostate cancer, ovarian cancer, cervical cancer, or myelodysplastic syndrome.

14. A method of inhibiting Hsp90, the method comprising administering a therapeutically effective amount of a composition of claim 1.
